(19) United States
(12) Patent Application Publication
(10) Pub. No.: US 2005/0203560 A1
(43) Pub. Date: Sep. 15, 2005

(54) Atmospherically activated thermal acupuncture needle

(76) Inventor: Christopher Eric Lieberman, Lansdowne, VA (US)

Correspondence Address: Christopher Lieberman, 19096 Icehouse Terrace, Lansdowne, VA, 20176, US

(21) Appl. No.: 10/798,802

(22) Filed: Mar. 12, 2004

(51) Int. Cl. A61B 17/34

(52) U.S. Cl. 606/189

(57) ABSTRACT

The present invention relates an improved acupuncture needle having a chemical coating of an oxidation exothermic type. The coating is applied to the acupuncture needle prior to packaging such that, during shipment and storage, the acupuncture needle and coating are isolated from atmospheric oxygen. At time of treatment, the packaging container is opened, allowing the chemical coating to be exposed to atmospheric oxygen such that an exothermic reaction takes place. The needle is inserted into the patients skin and the heat generated from the exothermic reaction is transferred through the needle and into the patient by means of conduction.

### CROSS-REFERENCE TO RELATED APPLICATIONS

Not applicable

### BACKGROUND

1. Field of Invention

The present invention relates to acupuncture therapy.

2. Description of Prior Art

The procedure of augmenting the therapeutic benefits of acupuncture with heat therapy is well known. In this process, known as moxibustion, a small of amount of dried leaves of Artemisia Vulgaris, or wormwood, are attached to the top portion of the acupuncture needle and burned once the needle has been inserted in the patient. An alternative form is to hold the lighted end of a cigar-shaped roll of the dried leaves against the needle causing it to heat up. In each case, the intent is to create heat that is transferred through the needle by means of conduction, and then into the specific acupuncture point on the patient. While moxibustion is intended to augment the positive effects of acupuncture, its use suffers from a number of disadvantages:
(a) To generate the positive effects of moxibustion, the dried leaves need to be ignited to produce heat. This process generates a large amount of smoke that both smells and is irritating to the eyes and sinuses of the patient and practitioner.
(b) Moxibustion can generate extremely high temperatures that can burn the patient leaving painful scars. This intense heat can also cause damage to both the nerves and calcium-ion channels at specific acupuncture points.
(c) Once the dried leaves are ignited, the temperature is difficult to regulate over a specific period of time. As a result, generating consistently repeatable results from one patient to the next is difficult, which inhibits the treatment process.
(d) Artemisia Vulgaris is generally purchased separately from acupuncture needles. As such it needs to be inventoried to ensure that a supply is available at treatment time. An inventory shortage could prevent heat therapy from being offered.

Prior art inventions have attempted to offer alternatives to the problems associated with moxibustion, however all have limitations. Most of these alternatives have been based on using electricity as the means of generating thermal energy. For example, U.S. Pat. No. 6,346,103 details a “heating acupuncture needle” that generates its heat by means of electrical impedance. The device requires a multi-step manufacturing process and an external energy source in the form of a battery of power generator. These requirements make such an invention cost-prohibitive in the highly commoditized market for acupuncture needles. Other designs have also have also incorporated electricity as a means of generating heat suffer from similar disadvantages. For example U.S. Pat. No. 3,938,526 details an invention that uses electrical energy to heat an acupuncture needle. This design also requires the use of an external source of electricity increasing both the cost and complexity of using such a solution.

### SUMMARY

The present invention relates to an acupuncture needle having a means of generating heat by means of an oxidation reaction when exposed to atmospheric oxygen.

Objects and Advantages

Accordingly, besides the objects and advantages of the enhanced therapeutic acupuncture needle described in my above patent, several objects and advantages of the present invention are:
(a) to provide an enhanced thermal acupuncture treatment that does not require burning and therefore does not produce smoke that is an eye and sinus irritant.
(b) to provide an enhanced thermal acupuncture needle in which the amount of thermal energy transferred to the patient can be properly controlled thereby ensuring that the patient does not suffer burns, or damage to the nerves or calcium-ion channels.
(c) to provide an enhanced acupuncture needle who's thermal properties can be made to be consistent from patient to patient
(d) to provide an enhanced thermal acupuncture treatment in which the acupuncture needle and means of generating thermal energy are integral to each other thereby ensuring that heat therapy is available when needed

Further objects and advantages are to provide an atmospherically activated thermal acupuncture needle that is easy to use, and both cost effective to manufacture and purchase.

### DRAWING FIGURES

FIG. 1 is a perspective view of the acupuncture needle completely enclosed within the packaging container;

FIGS. 2A and 2B are perspective views of the acupuncture needle and packaging container of FIG. 1 with one of the covering layers peeled back to expose the acupuncture needle;

FIGS. 3A, and 3B are perspective views of the acupuncture needle covered to varying degrees with atmospherically activated exothermic coating. FIG. 3C is a perspective view of the acupuncture needle with the atmospherically activated exothermic coating wrapped in a breathable material.

FIGS. 4A, 4B and 4C are cross-sectional rear views of the acupuncture needle with atmospherically activated exothermic coating.

### Reference Numerals In Drawings

10—packaging container
20—opposing sheet 1 of packaging container
22—side edge 1 of sheet 1
24—side edge 2 of sheet 1
26—end edge 1 of sheet 1
28—end edge 2 of sheet 1
30—opposing sheet 2 of packaging container
32—side edge 1 of sheet 2
34—side edge 2 of sheet 2
36—end edge 1 of sheet 2
38—end edge 2 of sheet 2
40—flap of sheet 1
42—flap of sheet 2
50—acupuncture needle
52—insertion end of acupuncture needle
54—shaft of acupuncture needle
60—atmospherically activated exothermic coating
62—sleeve covering exothermic coating
64—non-removable breathable material surrounding exothermic coating

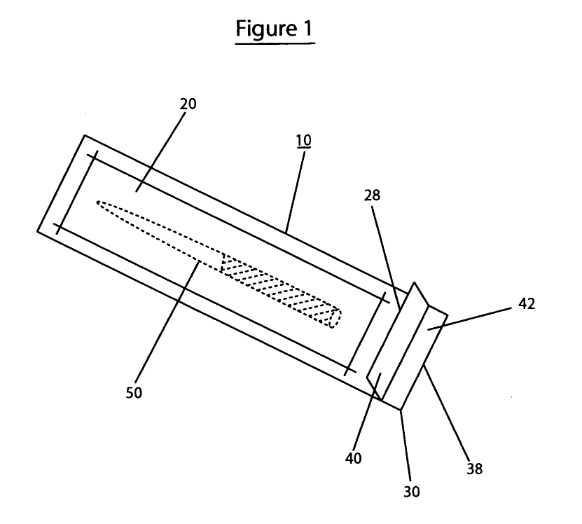

### DESCRIPTION—FIGS. 1, 2, 4, 6—PREFERRED EMBODIMENT

The nature and objects of the invention may be best understood by reference to the accompanying illustrative drawings taken in conjunction with the following detailed description. A preferred embodiment of the present invention is illustrated in FIGS. 1, 2A, 3A (perspective view), and 4A (cross-sectional rear view).

FIG. 1 is a perspective view showing the novel acupuncture needle 50 completely enclosed in a packaging container 10. The packaging container 10 is composed of non-breathable opposable sheets 20 and 30 that are arranged with their respective end and side edges in superposition. The opposable sheets 20 and 30 are sealed in such a way as to create a sterile internal environment for acupuncture needle 50. The end edges 28 and 38 are unsealed such that flaps 40 and 42 are created.

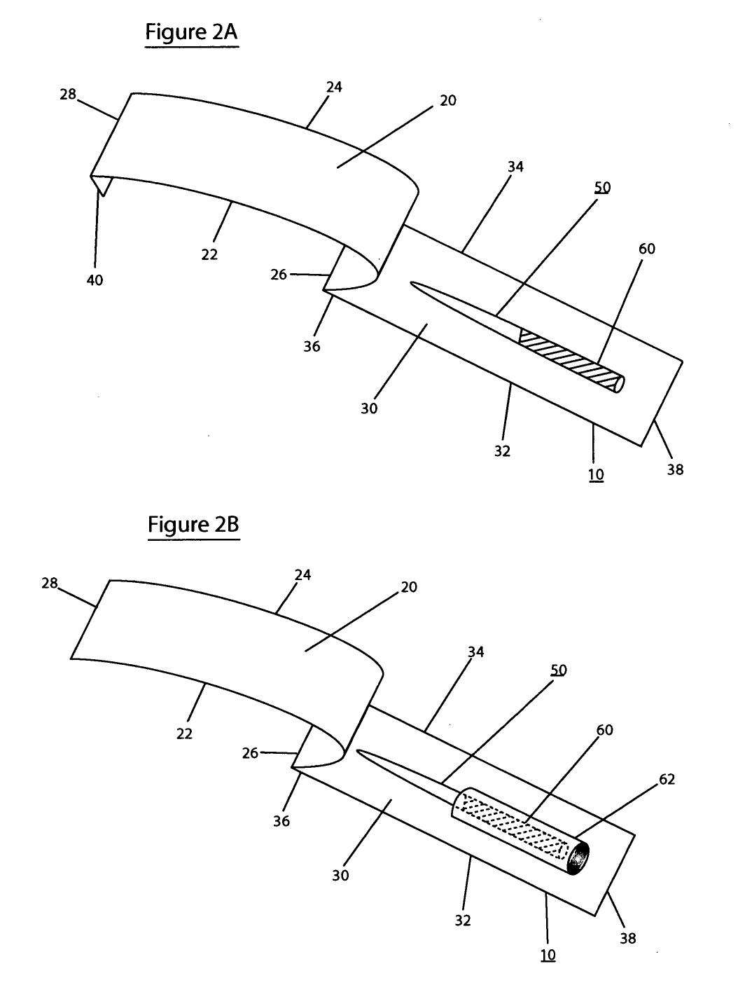

In FIG. 2A, the opposable sheets 20 and 30 of packaging container 10 are separated such that the novel acupuncture needle 50 is exposed. Sheet 20 has opposed side edges 22 and 24 and opposed end edges 26 and 28. Sheet 30 has opposed side edges 32 and 34 and opposed end edges 36 and 38.

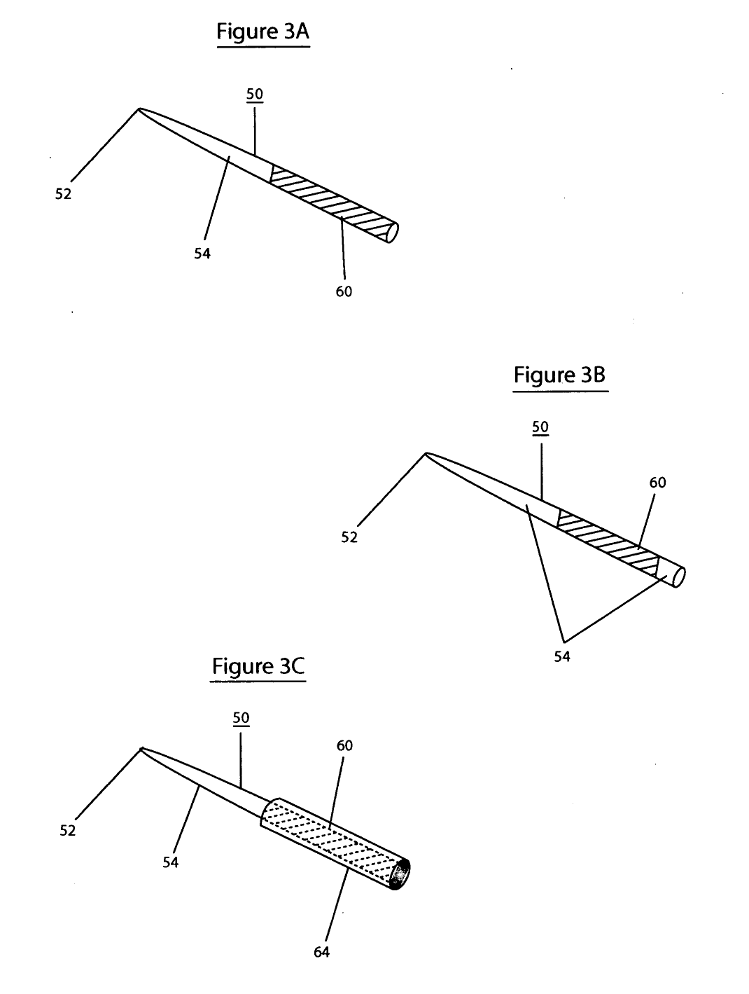

FIG. 3A shows a perspective view of the acupuncture needle 50 comprising an insertion end 52, a shaft 54, and an atmospherically activated exothermic coating 60. The atmospheric exothermic coating 60 is made from a compound capable of participating in an oxidation reaction upon exposure to atmospheric oxygen. Furthermore, the exothermic coating 60 can be applied to the acupuncture shaft 54 by means of spraying, dipping, rolling or similar application techniques that can be applied in a commercially viable manufacturing process.

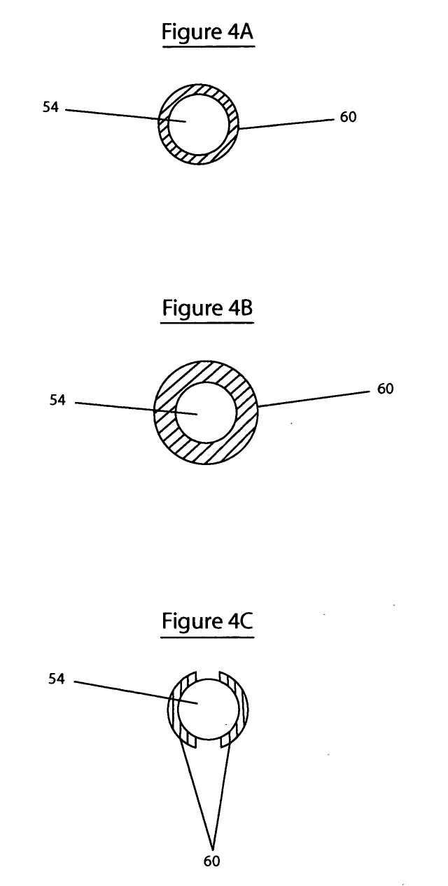

FIG. 4A shows a cross-sectional rear view of acupuncture needle 50 having a shaft 54 and an atmospherically activated exothermic coating 60.

Additional Embodiments—FIGS. 2B, 3B, 3C, 4B and 4C

Additional embodiments are shown in FIGS. 2B, 3B, 3C, 4B and 4C. In FIG. 2B, the opposing sheets 20 and 30 are shown without having flaps indicating that when arranged in superposition, they are sealed to each of their ends, respectively. FIG. 2B additionally shows an alternative embodiment to packaging the acupuncture needle 50. In this embodiment, the acupuncture needle 50 is at least partially encased in a sleeve 62, preferably surrounding the atmospherically activated exothermic coating 60. FIG. 3C shows an alternative embodiment where the atmospherically activated coating 60 is wrapped in a breathable material 64 that stays on acupuncture needle 50 even after it has been inserted in the patient. This would help slow the oxidation reaction while simultaneously insulating the acupuncture needle 50 such that the transfer of thermal into the shaft 54 is maximized. Additionally, the breathable material 64 can be used in conjunction with sleeve 62.

In FIGS. 3B, 4B and 4C, an alternative embodiment with regards to the atmospherically activated exothermic coating 60 is shown. FIG. 3B is a perspective view showing varying coverage of exothermic coating 60 along the length of acupuncture shaft 54. FIG. 4B is a cross-sectional rear view showing varying thickness of coverage of the exothermic coating 60 around the circumference of the acupuncture needle shaft 54. FIG. 4C is a cross-sectional rear view showing varying continuity of coverage of the exothermic coating 60 around the circumference of the acupuncture needle shaft 54.

Advantages

From the description above, a number of advantages of the atmospherically activated thermal acupuncture needle become evident:
a) The use of a coating capable of oxidizing upon exposure to atmospheric oxygen will generate sufficient thermal energy as to obviate the need to use moxibustion that is an irritant to both the eyes and sinuses when burned.
b) The use of an atmospherically activated coating ensures better temperature control thereby reducing the risk of skin burns and damage to the nerves and calcium-ion channels typical with moxibustion.
c) The use of an acupuncture needle whose means of generating thermal energy is applied during the manufacturing process ensures more precise levels of heat generation that in turn allows for more consistent treatment from one patient to the next.
d) The use of an acupuncture needle whose means of generating thermal energy is applied during the manufacturing process ensures that heat therapy is available to the patient when needed.

Further objects and advantages are to provide an acupuncture needle that is easy to use and cost effective to both manufacture and purchase.

Operation—FIGS. 1, 2 and 4

The manner of using the novel acupuncture needle 50 begins by pulling opposing sheets 20 and 30 apart using flaps 40 and 42. This opens the packaging container 10 allowing for the acupuncture needle to be removed. The acupuncture needle 50 can then be inserted into the patient. Alternatively, the sleeve 62 can be left on the acupuncture needle 50 until it is inserted into the patient, at which time the sleeve 62 can then be removed. This exposes the exothermic coating 60 to atmospheric oxygen 70, thereby initiating the oxidation reaction. As this reaction proceeds, heat will be generated which can be transferred through the acupuncture shaft 54 and into the patient. Generally acupuncture needles are left in the patient for between 10-30 minutes. This is a sufficient time for the oxidation reaction to near completion and consequently cool off allowing for the needle to be safely removed from the patient.

Conclusion, Ramification, and Scope

Thus the reader will see that the enhanced acupuncture needle of the invention provides a means of augmenting traditional acupuncture with a means of heat therapy that is safe, effective, easy to use, and cost-efficient to manufacture. While the above description contains much specificity, these should not be construed as limitations on the scope of the invention, but rather as an exemplification of one preferred embodiment thereof. Many other variations are possible. For example, the packaging container could take alternative shapes from the arrangement set forth, such as being formed as a cylinder or box. Accordingly, the scope of the invention should be determined not by the embodiments illustrated, but by the appended claims and their legal equivalents.

1. A disposable atmospherically activated thermal acupuncture needle comprising:
a) a tubular metal body
b) an atmospherically activated exothermic coating
c) a packaging container

2. an atmospherically activated exothermic coating according to claim 1, that can be applied to directly to said tubular metal body

3. an atmospherically activated exothermic coating according to claim 1, that can be applied to said tubular metal body before insertion into said packaging container

4. a packaging container according to claim 1, that encloses said acupuncture needle such that said coating is not exposed to atmospheric elements until said packaging container is opened

5. an atmospherically activated exothermic coating according to claim 1, in which when said packaging container is opened, is exposed to atmospheric oxygen such that an oxidation reaction occurs

6. an atmospherically activated exothermic coating according to claim 1, in which when said packaging container is opened, is exposed to atmospheric oxygen such that an exothermic reaction occurs

7. a tubular metal body according to claim 1, through which the heat generated from said exothermic reaction can be transferred to the patient
